(19) United States
(12) Patent Application Publication
(10) Pub. No.: US 2002/0146469 A1
(43) Pub. Date: Oct. 10, 2002

(54) Methods for reducing chronic stress in mammals

(76) Inventors: Benjamin Wiegand, Newtown, PA (US); Laura McCulloch, Cedar Knolls, NJ (US); Kathryn Dean, Ringoes, NJ (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/012,627

(22) Filed: Dec. 7, 2001

Related U.S. Application Data
(60) Provisional application No. 60/256,813, filed on Dec. 20, 2000.

(51) Int. Cl. A61N 1/30; A61K 35/78

(52) U.S. Cl. 424/725; 604/20

(57) ABSTRACT

This invention relates to methods for reducing chronic stress in mammals by administering a sensory regimen which reduces or down-regulates the activity of the HPA axis. The activity of the HPA axis of the mammal may be downregulated by at least one of the following methods: (1) reducing the amount of total daily adrenocortical hormone; (2) reducing adrenocortical hormone at any time point in the period from about 4 to about 8 hours following morning waking; (3) reducing the total daily adrenocortical hormone minus the integrative measure of morning peak adrenocortical hormone. Generally, the sensory regimen is selected from the group consisting of auditory stimuli, visual stimuli, tactile stimuli, gustatory stimuli, olfactory stimuli and combinations thereof.

### CLAIM OF PRIORITY

This application claims priority to U.S. patent application Ser. No. 60/256,813, filed Dec. 20, 2000, the disclosure of which is hereby incorporated by reference.

### FIELD OF THE INVENTION

This invention relates to methods for reducing chronic stress in mammals by administering a sensory regimen which reduces or down-regulates the activity of the hypothalamus-pituitary-adrenal (HPA) axis.

### BACKGROUND OF THE INVENTION

Advances in technology in the last century have brought benefits to society but have resulted in greater prevalence of stress in the daily lives of people at all levels of society. Our stress response mechanisms have not adapted at the same pace as advancing technology. The effect of stress on health and well being is well documented in “Why Zebra's Don't Get Ulcers—An Updated Guide to Stress, Stress Related Diseases and Coping,” Chapter 1, by Robert M. Sapolsky, ISBN 0-7167-3210-6 and by George P. Chrousos and Philip W. Gold in “The Concepts of Stress and Stress System Disorders—Overview of Physical and Behavioral Homeostasis”, JAMA, Mar. 4, 1992, Vol. 267, No. 9. For example, it is known that stress can cause or aggravate many conditions including immunosuppression and vulnerability to infectious diseases, gastric conditions, sleep problems, depression, premature birth in expectant mothers, low birth weight, degeneration of brain neurons leading to memory and learning problems, elevated blood pressure, heart complications and stroke due to elevated blood lipid levels and other health complications.

The region in the brain known as the hypothalamus drives the activity of the mammalian stress response. Specifically, the hypothalamus drives the production of “stress hormones” including catecholamines and glucocorticoids. The hypothalamus responds to a stressor by activating the sympathetic nerve endings in the adrenal medulla to produce adrenaline. The hypothalamus produces corticotrophin-releasing hormone (“CRH”) which acts upon the pituitary to release adrenocorticotrophic hormone (“ACTH”) which in turn acts upon the adrenal cortex to promote the production of cortisol. The CRH and sympathetic systems participate in a positive feedback loop so that activation of one system activates the other. Since increased cortisol secretion is an indication that the HPA (“HPA”) axis has been activated, conversely, a decrease in cortisol secretion would indicate a downregulation of HPA axis activity.

While in the short term, the activation of these physiological responses to stress can have beneficial and even life saving merits; long-term or chronic stress has negative effects on health and well being. If the physiological response to chronic stress is to lead to elevated production of stress hormones, in effect resetting their basal levels, then it could be hypothesized that sustained reduction of these hormones, namely resetting the basal levels to a lower value, would be beneficial in managing stress and promoting well being. Also, as these hormones act upon each other in a positive feedback loop, downregulation of one system would be expected to downregulate the other.

Resetting the basal levels of these stress hormones to a lower value could provide benefits including reduced perceived stress; reduced immunosuppression and vulnerability to infectious diseases; reduced incidence of gastric conditions; reduced incidence of sleep problems; reduced incidence of depression; reduced incidence of premature birth; reduced incidence of low birth weight; reduced incidence of degeneration of brain neurons leading to memory and learning problems; reduced incidence of elevated blood pressure; reduced incidence of heart complications and stroke due to elevated blood lipid levels; reduced deleterious effects on metabolism and reproduction; reduced incidence of abdominal adiposity; reduced contribution to aging; reduced incidence of addictive behaviors; and reduced occurrence of other health and behavioral complications that are caused or aggravated by stress.

A good measure of the reactivity of the HPA axis is a measure of adrenocortical activity. An adrenocortical hormone that can be easily measured is cortisol, which can be found in the blood, urine and the saliva of human beings. Cortisol is produced in the adrenal cortex and is involved in a number of neurological events. Some have found that the level of this hormone rises when an individual is subjected to psychological and/or physiological stress. See Kirschbaum, C. & Hellhammer, D. H., “Salivary Cortisol in Psychoendocrine Research: Recent Developments and Applications”; Psychoendocrinology, Vol. 19 No. 4, 1994, pp. 313-333. Methodology to accurately measure this adrenocortical 110(D hormone has been developed and refined over the past decade and is now applicable to measure HPA axis activity.

It has been recognized by those skilled in the art that a stressor induces an increase in the level of cortisol that is detectable in saliva. Reports of elevated salivary cortisol in response to psychological and physiological stress are reported by Kirschbaum, C. & Hellhammer, D. H., “Salivary Cortisol in Psychoendocrine Research: Recent Developments and Applications”; Psychoneuroendocrinology, Vol. 19 No. 4, 1994 pp. 313-333.

Others have found that when adults are subjected to psychological stress (practicing arithmetic under stressful conditions) that their level of stress can be monitored by their salivary cortisol, see JP Patent No.11-19076. The same researchers have shown that if the same individuals were exposed to certain fragrances before the stressful event, their level of salivary cortisol levels would not be as high as when they were psychologically challenged without the fragrance.

This study showed that not all fragrances were effective at reducing the stress-induced release of cortisol. Fragrances with lavender oil or mint oil successfully attenuated the stress induced increase in cortisol levels, while the fragrance with skatole had the opposite effect.

Others also describe the usefulness of fragrances in reducing the stress release of adrenocortical hormones. Japanese Patent Application No. JP9227399,entitled “An Adrenocortical Hormone Secretion Inhibitory Agent” and relates to an adrenocortical hormone secretion inhibitory agent comprising of the essence of the plants of the labiatae family. The stress release of adrenocortical hormone is suppressed by inhaling the essences of the family of labiatae plants. According to this application, members of the labiatae plant family are useful in reducing the stress release of adrenocortical hormone. However, there is no mention of the benefits of changing or reducing basal levels of adrenocortical hormone.

Co-pending U.S. patent application Ser. No. 09/676,876, filed Sep. 29, 2000 entitled “Method For Calming Human Beings Using Personal Care Compositions” and is another invention in which adrenocortical hormone is reduced as a means to calm a human being. According to this application, specific methods and compositions are useful in reducing levels of adrenocortical hormone at the time that the method of the invention is practiced, and results in a short term calming experience in the user.

Many currently marketed fragrant cosmetic products claim to have a “calming”, “stimulating” or “relaxing” benefit to the user. Typically, these products possess fragrances that are purported to deliver these benefits. To support these claims, several methods have been employed to measure the effects of fragrance on physiological parameters with varying degrees of success and unfortunately, much of the evidence for these purported benefits is the subject of folklore, rather than science.

Measures of salivary cortisol have been used in this disclosure to demonstrate the downregulation of endocrine parameters in the stress response system and to relate this physiological downregulation to a reduction in perceived stress. This downregulation of the HPA axis, as measured by cortisol reduction, is sufficient to reset basal levels. It is important to note that while pharmaceutical interventions are effective in downregulating the activity of the HPA axis, they require treatment by a medical professional and as such are not available to the public at large but usually limited to people who are have been identified by the medical community as being particularly vulnerable to stress.

### SUMMARY OF THE INVENTION

The invention relates to a method for reducing chronic stress in a mammal by downregulating the activity of the HPA axis by administering to said mammal an effective amount of a sensory regimen. The activity of the HPA axis of the mammal may be downregulated by at least one of the following methods: (1) reducing the amount of total daily adrenocortical hormone; (2) reducing adrenocortical hormone at any time point in the period from about 4 to about 8 hours following morning walking; (3) reducing the total daily adrenocortical hormone minus the integrative measure of morning peak adrenocortical hormone. Generally, the sensory regimen is selected from the group consisting of auditory stimuli, visual stimuli, tactile stimuli, gustatory stimuli, olfactory stimuli and combinations thereof.

### BRIEF DESCRIPTION OF THE DRAWINGS

FIG. 1 is a graph demonstrating the adrenocortical hormone in a mammal in the period from about 4 to about 8 hours following morning waking.

FIG. 2 is a graph demonstrating the total daily adrenocortical hormone.

FIG. 3 is a graph demonstrating the total daily adrenocortical hormone minus the morning peak.

### DETAILED DESCRIPTION OF THE INVENTION

The invention relates to a method for reducing chronic stress in a mammal by administering to said mammal a sensory regimen, which reduces or down-regulates the activity of the HPA axis by an amount sufficient to reset the basal activity of the HPA axis. Activity of the HPA axis is a measure of adrenal function.

As used herein, “mammals” include any of a class of warm-blooded higher vertebrates that nourish their young with milk secreted by mammary glands and have skin usually more or less covered with hair, and non-exclusively includes humans, dogs and cats.

The term “effective amount” refers to the duration of the sensory regimen sufficient to create the desired response, i.e., reduction or down-regulation of the activity of the HPA axis. The effective amount will vary with the age, physical, and emotional condition of the mammal being treated, the nature of concurrent therapy, the specific regimen employed, and like factors.

The term “amount sufficient to reset the basal activity of the HPA axis” refers to the reduction in HPA activity that is needed to lower overall activity of the PAPA axis. Examples of desired responses include reduction of any of the following: (1) adrenocortical hormone at any time point in the period from about 4 to about 8 hours following morning waking; (2) the amount of total daily adrenocortical hormone: (3) total daily adrenocortical hormone minus the integrative measure of morning peak adrenocortical hormone.

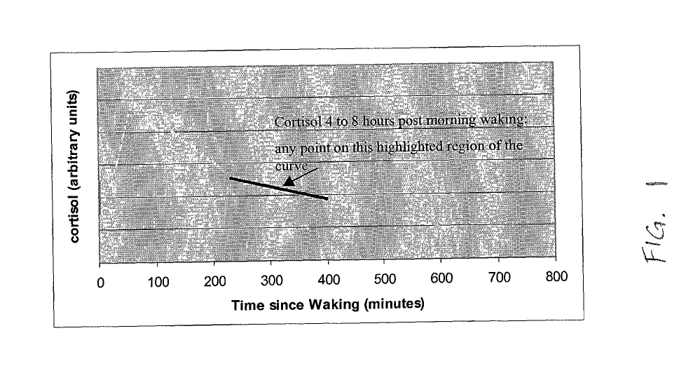

As used herein, the term “adrenocortical hormone in a mammal in the period from about 4 to about 8 hours following morning waking” refers to the amount of adrenocortical hormone secreted at ally point in the 4 to 8 hours following morning waking, in any increments of time, for example minutes and hours. Any point on this region of the curve is included in this definition. The region on the curve representing the 4 to 8 hours following morning waking of the adrenocortical hormone cortisol in saliva as a function of time since morning waking is illustrated in FIG. 1.

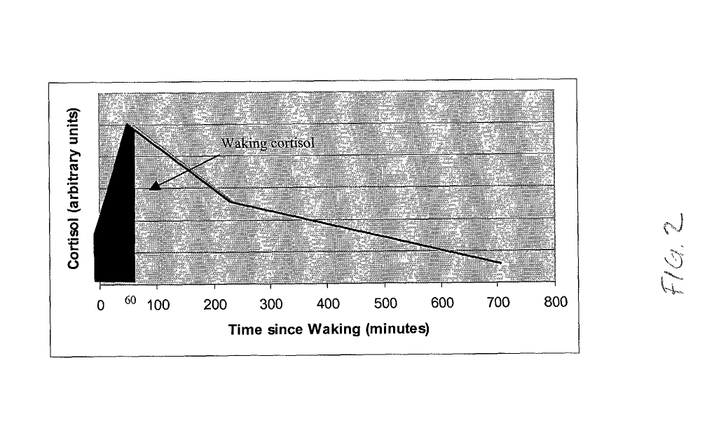

As used herein, the term “total daily adrenocortical hormone” refers to the total amount of adrenocortical hormone secreted throughout the wakeful period in a 24 hour period typically divided into a period of wakefulness and a period of sleepfulness. The most substantial amount of adrenocortical hormone secreted by an individual during the wakeful period of a 24-hour day is typically secreted in the first 12 hours immediately following morning waking. The area under the curve (“AUC”) of salivary cortisol secretion as a function of time since waking for the 12 hour period following morning waking is illustrated in FIG. 2 and is used in examples in this disclosure to represent the total amount of cortisol secreted throughout the wakeful period of a 24 hour day.

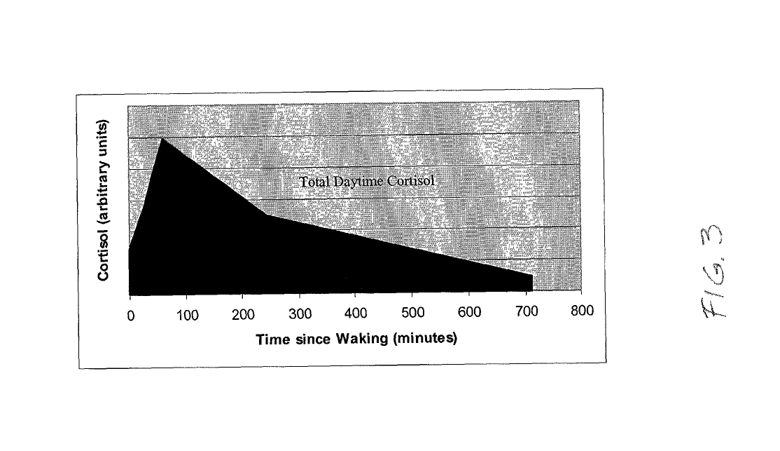

As used herein, the term “total daily adrenocortical hormone minus the integrative measure of morning peak adrenocortical hormone” refers to the total amount of adrenocortical hormone secreted throughout the wakeful period in a 24 hour period typically divided into a period of wakefulness and a period of sleepfulness, as defined above, having subtracted the area under the morning peak. These areas are illustrated for the adrenocortical hormone cortisol in saliva in FIG. 3.

In another embodiment, the invention relates to a method of reducing basal levels of stress hormones in a mammal by administering to said mammal an effective amount of a sensory regimen, wherein stress hormones are defined as adrenocortical hormones and catecholamines.

The invention relates to a method of reducing chronic stress in mammals by affecting adrenal functions such as to reduce HPA activity. It has been previously shown that a good measure of the reactivity of the HPA axis is a measure of adrenocortical activity. Cortisol, an adrenocortical hormone, is a good representative marker for adrenocortical activity, and methodology to measure its level has been developed over the last decade. Cortisol is found in a number of different fluids in the body, including serum, saliva and urine. Recently it has been shown that cortisol measures done in saliva samples can be correlated with serum samples and do not have the associated concerns with serum measurements. See, E. Aardal and A. Hohm, J. Clin. Chem. Clin. Biochem. 33:927-932, 1995. Firstly, cortisol collection methodology in serum requires a pinprick, needle, or other device to collect the fluids, which of itself can cause a stressful response. Use of intravenous devices for long term collections are possible, but affect the individuals Quality of Life and are therefore not totally representative of their normal response. Secondly, it is well known that the majority of cortisol in serum is bound to corticosteroid-binding globulin (CBG), albumin and erythrocytes (85% -98%). As it is only the free, unbound cortisol that would be expected to impart any physiological effect, it is important to measure this parameter. Urinary cortisol measurements are also possible, however, this would represent a more integrative measure over time, instead of a momentary measure, which is important to better understand the stress profile of the individual.

In saliva, much of the cortisol found is free, making it a sensitive measure. If cortisol is reduced sufficiently and the reduction is sustained over a sufficient period of time, then the quality of life of an individual may be improved.

Using total daily cortisol (cortisol secreted throughout the wakeful period in 24 hour period typically divided into a period of wakefulness and a period of sleepfuleness) as an index of HPA activity, total daily cortisol should be reduced by 5-50% and more preferably by 10-40% and most preferably by 15-30% from the amount secreted on a typical day in which no relaxation regimen has been practiced.

Cortisol follows a diurnal rythym with the profile typically exhibiting a morning peak approximately 30 to 45 minutes following waking. The total daily adrenocortical hormone minus the integrative measure of morning peak adrenocortical hormone (as calculated by the area under the curve minus the area under the morning peak) is yet another useful index of HPA activity. This value should be reduced by 5-70% and more preferably by 10-60% and most preferably by 20-50% from the amount secreted on a typical day in which no relaxation regimen has been practiced.

Another useful index of the activity of the HPA axis is the cortisol level in saliva about 4 hours to about 8 hours following waking, preferably about 4 hours following morning waking. if this level is sufficiently reduced from its baseline value then the quality of life of an individual may be improved. Cortisol 4 hours post waking should be reduced by 5-70% and more preferably by 10-60% and most preferably by 20-50% from the amount secreted on a typical day in which no relaxation regimen has been practiced.

Stimuli used to provide the sensory regimen generally are those which provide an experience which the individual who intends to practice the invention finds pleasant. The sensory regimen can be any regimen that is relaxing to the user. Generally, the sensory regimen is selected from the group consisting of auditory stimuli, visual stimuli, tactile stimuli, gustatory stimuli and olfactory stimuli, and combinations thereof.

Suitable auditory stimuli include, but are not limited to, music and sounds of nature that are soothing or relaxing to the consumer. The term music is used herein to include instrumental and lyrical compositions; tunes; melodies; harmonies; songs; beats and frequencies such as those from metronomes, tuning forks, bells, beat machines, chimes; poetry and rhymes. The music may be of any genre, including, but not limited to, classical, soft rock, easy listening, progressive, country, and show tunes. The sounds of nature include, but are not limited to, animal sounds, such as whales singing or birds chirping; insect sounds, such as crickets; and sounds of the environment, such as a running stream or a waterfall. Sounds that have consistently soft dynamics with minimal melodic and harmonic variability, having little or no conventional beat pitch, little or no vocal, slow tempo, little or no percussion or strong rhythm are particularly effective in relaxing or soothing the user. Sounds that use a binaural beat created by using two pure frequencies, usually one in each ear, are useful in improving the mood of the user. Binaural beats in the frequency range of delta, theta and alpha brain wave frequencies are useful for relaxing the user and beats in the frequency range of beta wave activity are useful for promoting mental alertness in the user. The auditory stimuli may include, but are not limited to, a cassette tape, videotape, compact disc, interactive toys and games, websites, and a computer audio file.

The visual stimuli may include, but are not limited to, soft lights, candles, videos, movies, paintings, murals, books, landscapes, interactive toys and games, websites, and computer image files that are soothing or relaxing to the consumer. The soft lights may be of any color, such as blue, green, pink, purple, and the like. Cool colors, such as blue and green hues, are preferred to soothe the user and aid relaxation; and warmer colors, such as oranges and reds are preferred to uplift the user. Pastel shades, which are low saturation hues, are useful in soothing the user. The light may be provided in the kit as a bulb, which can be inserted into a lamp at home, or may be provided in the kit as a lamp. Lights that utilize fiber optics may also be useful in the kits of this invention. The fiber optic lights may, as is known in the art, change colors intermittently. Soft lighting of approximately 500 lux is useful in relaxing the user, particularly in the evening hours prior to bedtime. Bright light of around 2000 lux or greater is useful in improving the mood of the user when used in the wakeful period of the day such as at awakening or any other time during the day prior to the few hours preceding bedtime.

Combinations of light and sound that have frequency patterns in the range of delta, theta and alpha brain wave frequencies are useful for relaxing the user and those that have patterns in the frequency range of beta wave activity are useful for promoting mental alertness in the user.

The tactile stimuli useful in the present invention includes, but is not limited to, computer software, interactive toys and games, bubble baths, lotions, and personal care compositions. “Personal care compositions” refers to personal cosmetic, toiletry, and healthcare products such as wipes, washes, baths, shampoos, gels, soaps, sticks, balms, sachets pillows, mousses, sprays, lotions, creams, cleansing compositions, powders, oils, bath oils and other bath compositions which may be added to a bath. Personal care compositions may also include, but are not limited to, aerosols, candles, and substances that may be used with vaporizers. The aforementioned wipes, washes, baths, shampoos, gels, soaps, sticks, balms, sachets, pillows, mousses, sprays, lotions, creams, cleansing compositions, oils, bath oils, aerosols, candles and substances which may be used with vaporizers are commercially known to those who have a knowledge of preparing personal care compositions. One example of a suitable personal care composition is Johnson's Bedtime Bath®.

The computer software may be of an interactive nature, such that the consumer relaxes while utilizing the software. Such software includes video games, crossword puzzles and the like.

Gustatory experiences also help reduce stress. Therefore, the method of the invention may include food and beverages, such as, but not limited to, fruits, candies, crackers, cheese, teas, and the like.

The method of the invention may also include olfactory sensory experiences, such as fragrances. Fragrances that the user finds pleasant and to have a calming effect on their mood are useful in the practice of this invention. Suitable fragrances include relaxing fragrances, but are not limited to those perfume compositions described in UK application 0031047.4 the disclosure of which is hereby incorporated by reference. Also suitable are the fragrances described in co-pending U.S. patent application Ser. No. 09/676,876, filed Sep. 29, 2000 entitled “Method For Calming Human Beings Using Personal Care Compositions”, the disclosure of which is hereby incorporated by reference. Generally, the fragrance can be any fragrance that is perceivable and relaxing to the user and will downregulate the activity of the HPA axis. Suitable fragrances include relaxing fragrances, including but not limited to those relaxing fragrances available from Quest International, an example of which is PD 1861 and described in UK application 0031047.4. Also suitable are the fragrances described in co-pending U.S. patent application Ser. No. 09/676,876, filed Sep. 29, 2000 entitled “Method For Calming Human Beings Using Personal Care Compositions”, the disclosure of which is hereby incorporated by reference.

A preferred means of delivering sensory stimuli is in the form of a personal care composition. Personal care compositions are particularly useful in delivering olfactory stimuli. For example, the sensory fragrance may be produced by blending the selected essential oils and odoriferous components under ambient conditions until the final mixture is homogenous using equipment and methodology commonly known in the art of fragrance compounding. It is preferable to store the final sensory fragrance mixture under ambient conditions for a few hours after mixing before using it as a component of a personal care composition.

The personal care compositions of the present invention may then be produced by blending the desired components with the sensory fragrance using equipment and methodology commonly known in the art of personal care product manufacture. In order to improve the solubilization of the sensory fragrance in aqueous personal care compositions, the sensory fragrance may be pre-blended with one or more of the nonionic surfactants.

“Personal care compositions” refers to personal cosmetic, toiletry, and healthcare products such as dry and wet wipes, washes, baths, shampoos, gels, soaps, sticks, balms, sachets, pillows, mousses, sprays, lotions, creams, cleansing compositions, powders, oils, bath oils and other bath compositions which may be added to a bath. Personal care compositions may also include, but are not limited to, aerosols, candles, and substances that may be used with vaporizers. The aforementioned wipes, washes, baths, shampoos, gels, soaps, sticks, balms, sachets, pillows, mousses, sprays, lotions, creams, cleansing compositions, oils, bath oils, aerosols, candles and substances which may be used with vaporizers are commercially known to those who have a knowledge of preparing personal care compositions. Suitable personal care composition, include but are not limited to Johnson's Bedtime Bath.

In order to achieve the desired response in a mammal. the personal care composition may be used in a dosing amount that is in accordance with the prescribed directions of the personal care composition.

It is desirable to combine multiple sensory experiences useful for downregulating HPA activity and consequently reduce adrenocortical hormone below a baseline level. For example, a daily regime may include a fragrance; soft light; bubble bath containing fragrance; and relaxing music. The fragrance may be sniffed intermittently during the day while sitting in a softly lit room and listening to the relaxing music. The bubble bath containing fragrance may be used in the morning or at night when bathing or showering while listening to the relaxing music.

In a particularly preferred embodiment, the sensory regimen is administered daily for at least one week and comprises smelling a relaxing fragrance while soaking in a bath and listening to relaxing music. Further benefits are noticed when the sensory regimen includes soft lighting as described above.

Although a greater effect is generally achieved when multiple stimuli are used together, it should be obvious to one skilled in the art that a single exposure to an effective stimuli could be envisaged to have the same sustainable effect as multiple exposures to the stimuli described in the body of this invention and so are included in the invention.

As discussed above, it has been discovered that the administration of a sensory regimen can result in a reduction in the stress level of a mammal. It has been previously shown that pharmaceutically active CRH antagonists can provide similar benefits, however, there are resultant side effects that are prevalent when these active materials are used. In another embodiment of the. invention, the combination of the use of the sensory regimen and the CRH antagonist provides for a more potent treatment. In another embodiment, the combination of the use of the sensory regimen and the CRH antagonist allows for a lower dose of the CRH antagonist to be used.

Examples of CRH antagonists include, but are not limited to Astressin, D-PheCRH (12-41), and alpha helical CRH (9-41), and others known in the art. In yet another embodiment, the methods according to the invention may be practiced in combination with the administration of pharmaceuticals that downregulate CRH, such as antidepressants including but not limited to selective serotonin reuptake inhibitors (SSRI), for example Prozac. Such pharmaceuticals should be administered in accordance with the directions prescribed by an authorized physician.

In order to illustrate the invention the following examples are included. These examples do not limit the invention. They are meant only to suggest a method of practicing the invention. Those knowledgeable in the calming of human beings as well as other specialties may find other methods of practicing the invention. Those methods are deemed to be within the scope of this invention.

### EXAMPLES

### Example 1

### One Time Exposure to Olfactory and Audio Stimuli and Short Term Effect on HPA Activity as Measured by Cortisol in Saliva

A group of males and females aged 20 to 55 in good general health were invited to participate in a study in which over the course of 10 minutes they would smell a fragrance that was subjectively perceived to be pleasant and relaxing while listening to soothing sounds. The purpose of this study was to measure the effect of the experience on HPA activity as measured by cortisol in saliva in the short time period following the experience.

Approximately 1 ml of saliva was collected in vials from each of the 10 male and female volunteers by having each adult drool or spit into an independent vial. The samples were stored in a refrigerator until later analyzed for cortisol concentration. The samples were collected and analyzed as per the method set forth in co-pending U.S. patent application Ser. No. 09/676,876, filed Sep. 29, 2000 entitled “Method For Calming Human Beings Using Personal Care Compositions”. Each adult was then asked to frequently smell a sorbarod containing a fragrance that is perceived to be a pleasant and relaxing fragrance identified as PD 1861 available from Quest International over a 10 minute period while listening to relaxing music from the music CD entitled “Relax with Ocean Relaxing Surf” by Eclipse Music Group. Twenty to thirty minutes later, following the 10 minutes in which each adult had smelled the fragrance and listened to the music a second saliva sample was collected from each adult in an independent vial and stored as set forth above.

The results of the cortisol analyses are presented in Table 1 below.

TABLE 1

| | Cortisol | Cortisol | |
|---|---|---|---|
| | Before | After | % |
| Panelist | (ug/dl) | (ug/dl) | Change |
| 1 | 0.266 | 0.255 | −4.0 |
| 2 | 0.388 | 0.265 | −31.6 |
| 3 | 0.193 | 0.279 | 44.2 |
| 4 | 0.261 | 0.169 | −35.2 |
| 5 | 0.315 | 0.257 | −18.4 |
| 6 | 0.351 | 0.181 | −48.3 |
| 7 | 0.233 | 0.160 | −31.5 |
| 8 | 0.317 | 0.255 | −19.7 |
| 9 | 0.179 | 0.111 | −37.9 |
| 10 | 0.233 | 0.299 | 28.3 |
| Mean | 0.274 | 0.223 | −15.4 |

The results indicate that the fragrance and music experience results in a 15% mean reduction in salivary cortisol, which indicates that the activity of the HPA axis has decreased in the short time period following the relaxing experience. this short term reduction in cortisol is useful in confirming that an experience is relaxing but does not answer whether or not there is a downregulation of the HPA axis over a period of time greater than the duration of the event studied in this example.

### Example 2-6

Three groups of women (Groups A-C) participated in a study in which mood and behavior self-assessments were made and saliva samples were collected at set time points throughout the day for the purpose of measuring cortisol.

In Example 2, Group A was exposed to a one time relaxing fragrance experience at a set point in the morning.

In Example 3, Group B was exposed to the same fragrance experience as in Group A but with multiple exposures through the day, including one prior to the onset of sleep.

In Example 4, Group C was exposed to the same fragrance as Groups A&B but was also exposed to relaxing music during the same period. Group C had multiple exposures to the music and fragrance at set time points throughout the day. At a set time prior to the anticipated onset of sleep, panelists in Group C bathed in a warm (about 33 to about 37° C.) tub with the same fragrance as experienced throughout the day, with music and low ambient lighting.

The fragrance and music stimuli used in Examples 2-6 was the same fragrance and music stimuli used in Example 1.

Within the tables containing the reported results, “NA” denotes not available, due to failure of panelist to collect sample, sample loss or contamination of sample,

### Example 2

### One Time Exposure to Fragrance (Group A)

A group of women aged 20-40 years and in good health (Group A) participated in an ambulatory study in their natural environment in which they were asked to collect approximately 1 ml of saliva by drooling or spitting into independent vials at set points throughout each day of the study for the purpose of measuring cortisol concentrations. These saliva samples were collected:

i) upon waking

ii) 30 minutes post waking

iii) 65 minutes post waking

iv) 4 hours post waking

v) 8 hours post waking

vi) 12 hours post waking

They were also asked to complete self-assessments of their mood and behavior. The study lasted for 5 days. Day 1 of the study served as the control day in which saliva samples were collected and questionnaires completed but no treatment regimen had been prescribed. On Day 2 of the study, the panelists were asked to smell a pleasant relaxing fragrance (PD 1861 from Quest International) for a period of 5 minutes, which occurred approximately 30 minutes after morning waking. On days 2-5 no treatment regimen was prescribed.

The salivary cortisol data for Example 2 Group A is presented in Tables 2-9 below.

TABLE 2
Group A Salivary Cortisol in Sample Collected 30 Minutes Post
Waking

| | Day 1 | Day 2 | Day 3 | Day 4 | Day 5 |
|---|---|---|---|---|---|
| Panelist | (ug/dl) | (ug/dl) | (ug/dl) | (ug/dl) | (ug/dl) |
| A-1 | NA | 0.284 | 0.175 | 0.178 | 0.162 |
| A-2 | 0.059 | 0.125 | 0.030 | 0.134 | 0.100 |
| A-3 | 0.675 | 1.113 | 0.518 | 0.487 | 0.821 |
| A-4 | 0.648 | 0.503 | 0.803 | 0.360 | 1.013 |
| A-5 | 0.550 | 0.401 | 0.209 | 0.740 | 0.404 |
| A-6 | 0.648 | 0.503 | 0.803 | 0.360 | 1.013 |
| A-7 | 0.321 | 0.646 | 0.515 | 0.655 | 0.671 |

TABLE 3
Group A Salivary Cortisol in Sample Collected 4 hours Post Waking

| | Day 1 | Day 2 | Day 3 | Day 4 | Day 5 |
|---|---|---|---|---|---|
| Panelist | (ug/dl) | (ug/dl) | (ug/dl) | (ug/dl) | (ug/dl) |
| A-1 | 0.081 | 0.069 | 0.102 | 0.109 | 0.224 |
| A-2 | 0.136 | 0.070 | 0.047 | 0.071 | 0.295 |
| A-3 | 0.061 | 0.044 | 0.084 | 0.045 | 0.050 |
| A-4 | 0.144 | 0.114 | 0.188 | 0.098 | 0.058 |
| A-5 | 0.061 | 0.386 | 0.508 | 0.149 | 0.293 |
| A-6 | 0.117 | 0.061 | 0.069 | 0.189 | 0.657 |
| A-7 | 0.502 | 0.274 | 0.120 | 0.277 | 0.260 |

TABLE 4
Group A Salivary Cortisol in Sample Collected 8 Hours Post Waking

| | Day 1 | Day 2 | Day 3 | Day 4 | Day 5 |
|---|---|---|---|---|---|
| Panelist | (ug/dl) | (ug/dl) | (ug/dl) | (ug/dl) | (ug/dl) |
| A-1 | 0.061 | 0.108 | 0.102 | 0.080 | 0.142 |
| A-2 | 0.154 | 0.309 | 0.040 | 0.185 | NA |
| A-3 | 0.095 | 0.086 | 0.064 | 0.010 | 0.069 |
| A-4 | 0.123 | 0.087 | 0.242 | 0.055 | 0.071 |
| A-5 | 0.302 | 0.093 | 0.660 | 0.464 | 0.221 |
| A-6 | 0.120 | 0.114 | 0.140 | 0.037 | 0.118 |
| A-7 | 0.105 | 0.233 | 0.252 | 0.105 | 0.149 |

TABLE 5
Group A Salivary Cortisol in Sample Collected 12 Hours Post Waking

| | Day 1 | Day 2 | Day 3 | Day 4 | Day 5 |
|---|---|---|---|---|---|
| Panelist | (ug/dl) | (ug/dl) | (ug/dl) | (ug/dl) | (ug/dl) |
| A-1 | 0.619 | 0.113 | 0.178 | 0.150 | 0.099 |
| A-2 | 0.307 | 0.093 | 0.053 | 0.079 | NA |
| A-3 | 0.038 | 0.042 | 0.053 | 0.013 | 0.062 |
| A-4 | 0.098 | 0.059 | 0.085 | 0.060 | 0.165 |
| A-5 | 0.062 | 0.050 | 0.562 | 0.123 | 0.083 |
| A-6 | 0.055 | 0.065 | 0.046 | 0.049 | 0.034 |
| A-7 | 0.124 | 0.085 | 0.076 | 0.133 | 0.138 |

TABLE 6
Group A Mean Cortisol Values

| Minutes Since | Day 1 | Day 2 | Day 3 | Day 4 | Day 5 |
|---|---|---|---|---|---|
| Morning Waking | (ug/dl) | (ug/dl) | (ug/dl) | (ug/dl) | (ug/dl) |
| 30 | 0.484 | 0.511 | 0.436 | 0.416 | 0.598 |
| 240 | 0.157 | 0.145 | 0.16 | 0.134 | 0.262 |
| 480 | 0.137 | 0.147 | 0.214 | 0.138 | 0.128 |
| 720 | 0.186 | 0.072 | 0.15 | 0.087 | 0.097 |

Day 1 is the control, day 2 fragrance in the morning, with no treatment occurring on Days 2-5. An integrative measure of cortisol calculated from the area under the curve for each day may be made. The values of the area under the curve (AUC) for Group A for each of the 5 days of the study are presented in Table 7 below.

TABLE 7
Total Area Under Curve of Salivary Cortisol for Group A

| | Total AUC |
|---|---|
| | (arbitrary |
| Day | units) |
| 1 | 130.0 |
| 2 | 156.4 |
| 3 | 151.1 |
| 4 | 117.4 |
| 5 | 164.1 |

The values of the AUC minus the peak for Group A for each of the 5 days of the study presented in Table 8 below.

TABLE 8
AUC Minus the Morning Peak Area of Salivary Cortisol for Group A

| | AUC Minus |
|---|---|
| | Morning Peak Area |
| Day | (arbitrary units) |
| 1 | 107.0 |
| 2 | 91.5 |
| 3 | 122.2 |
| 4 | 87.8 |
| 5 | 128.8 |

The mean cortisol values for Group A 4 hours post waking are presented in Table 9 below.

TABLE 9
Group Mean Cortisol 4 Hours Post Waking

| | Mean Cortisol 4 Hours Post Waking |
|---|---|
| Day | (ug/dl) |
| 1 | 0.157 |
| 2 | 0.145 |
| 3 | 0.160 |
| 4 | 0.134 |
| 5 | 0.262 |

### Example 3

### Multiple Exposures to Pleasant Relaxing Fragrance with Ambient Lighting (Group B)

A group of women aged 20-40 years and in good health (Group B) participated in an ambulatory study in their natural environment in which they were asked to collect approximately 1 ml of saliva by drooling or spitting into independent vials at set points throughout each day of the study for the purpose of measuring cortisol concentrations. These saliva samples were collected:

i) upon waking

ii) 30 minutes post waking

iii) 65 minutes post waking

iv) 4 hours post waking

v) 8 hours post waking

vi) 12 hours post waking

They were also asked to complete self-assessments of their mood and sleep behavior. The study lasted for 5 days. Day 1 of the study served as the control day in which saliva samples were collected and questionnaires completed but no treatment regime had been prescribed. On days 2-5 of the study, the panelists were asked to smell a pleasant relaxing fragrance (PD1861 from Quest International) while sitting comfortably in a room with low level of ambient lighting for a period of 5 minutes approximately 30 minutes after morning waking, 4 hours after waking and 8 hours after waking.

The salivary cortisol data for Example 3 Group B is presented in Tables 10-17 below.

TABLE 10
Group B Salivary Cortisol in Sample Collected 30 Minutes Post
Waking

| | Day 1 | Day 2 | Day 3 | Day 4 | Day 5 |
|---|---|---|---|---|---|
| Panelist | (ug/dl) | (ug/dl) | (ug/dl) | (ug/dl) | (ug/dl) |
| B-1 | 0.181 | 0.117 | 0.281 | 0.263 | 0.016 |
| B-2 | 0.233 | 0.039 | 0.016 | 0.023 | 0.013 |
| B-3 | 0.150 | 0.396 | 0.101 | 0.091 | 0.040 |
| B-4 | 0.601 | 1.088 | 0.825 | 1.630 | 1.998 |
| B-5 | 0.646 | 0.315 | 0.459 | 0.161 | 0.348 |

TABLE 11
Group B salivary Cortisol in Sample Collected 4 hours Post Waking

| | Day 1 | Day 2 | Day 3 | Day 4 | Day 5 |
|---|---|---|---|---|---|
| Panelist | (ug/dl) | (ug/dl) | (ug/dl) | (ug/dl) | (ug/dl) |
| B-1 | 0.096 | 0.165 | 0.228 | 0.175 | 0.112 |
| B-2 | 0.054 | 0.041 | 0.036 | 0.019 | 0.022 |
| B-3 | 0.075 | 0.076 | 0.130 | 0.074 | 0.080 |
| B-4 | NA | 0.832 | 1.028 | 1.138 | 0.667 |
| B-5 | 0.217 | 0.197 | 0.125 | 0.110 | NA |

TABLE 12
Group B salivary Cortisol in Sample Collected 8 hours Post Waking

| | Day 1 | Day 2 | Day 3 | Day 4 | Day 5 |
|---|---|---|---|---|---|
| Panelist | (ug/dl) | (ug/dl) | (ug/dl) | (ug/dl) | (ug/dl) |
| B-1 | 0.158 | 0.115 | 0.328 | 0.061 | 0.066 |
| B-2 | 0.039 | 0.050 | 0.032 | 0.019 | 0.015 |
| B-3 | 0.025 | 0.035 | 0.017 | 0.071 | 0.037 |
| B-4 | NA | 1.091 | 1.519 | 0.852 | 1.402 |
| B-5 | 0.028 | 0.040 | 0.048 | 0.017 | 0.014 |

TABLE 13
Group B Salivary Cortisol Sample Collected 12 hours Post Waking

| | Day 1 | Day 2 | Day 3 | Day 4 | Day 5 |
|---|---|---|---|---|---|
| Panelist | (ug/dl) | (ug/dl) | (ug/dl) | (ug/dl) | (ug/dl) |
| B-1 | 0.067 | 0.348 | 0.228 | 0.073 | 0.038 |
| B-2 | 0.031 | 0.041 | 0.041 | 0.023 | 0.011 |
| B-3 | 0.044 | 0.020 | 0.022 | 0.072 | 0.072 |
| B-4 | NA | 0.616 | NA | 0.308 | 0.832 |
| B-5 | 0.011 | 0.049 | 0.050 | 0.016 | 0.016 |

TABLE 14
Group B Mean Cortisol Values

| Minutes Since | | | | | |
|---|---|---|---|---|---|
| Morning | Day 1 | Day 2 | Day 3 | Day 4 | Day 5 |
| Waking | (ug/dl) | (ug/dl) | (ug/dl) | (ug/dl) | (ug/dl) |
| 30 | 0.362 | 0.391 | 0.336 | 0.434 | 0.483 |
| 240 | 0.111 | 0.262 | 0.309 | 0.303 | 0.183 |
| 480 | 0.063 | 0.266 | 0.389 | 0.204 | 0.307 |
| 720 | 0.038 | 0.058 | 0.085 | 0.098 | 0.194 |

Day 1 is the control, while beginning on Day 2 and continuing through Day 5, the panelist is exposed to fragrance at 3 time points throughout the day. An integrative measure of cortisol calculated from the area under the curve for each day may be made. The values of the area under the curve (AUC) for Group B for each of the 5 days of the study are presented in Table 15 below.

TABLE 15
Total Area Under Curve of Salivary Cortisol for Group B

| Day | Total AUC (arbitrary units) |
|---|---|
| 1 | 78.1 |
| 2 | 170.8 |
| 3 | 208.4 |
| 4 | 174.5 |
| 5 | 188.9 |

The values of the AUC minus the peak are for Group A for each of the 5days of the study are presented in Table 16 below.

TABLE 16
AUC Minus the Peak Area of Salivary Cortisol for Group B

| Day | AUC Minus Peak Area (arbitrary units) |
|---|---|
| 1 | 50.3 |
| 2 | 157.3 |
| 3 | 205.5 |
| 4 | 160.7 |
| 5 | 157.4 |

The mean cortisol for group B 4 hours post waking is shown in Table 17 below.

TABLE 17
Group Mean Cortisol 4 Hours Post Waking

| | Mean Cortisol 4 Hours Post Waking |
|---|---|
| Day | (ug/dl) |
| 1 | 0.111 |
| 2 | 0.262 |
| 3 | 0.309 |
| 4 | 0.303 |
| 5 | 0.220 |

### Example 4

### Multiple Exposures to Fragrance, Music and Ambient Lighting (Group C)

A group of women aged 20-40 years and in good health (Group C) participated in an ambulatory study in their natural environment in which they were asked to collect approximately 1 ml of saliva by drooling or spitting into independent vials at set points throughout each day of the study for the purpose of measuring cortisol concentrations. These saliva samples were collected as follows:

i) upon waking

ii) 30 minutes post waking

iii) 65 minutes post waking

iv) 4 hours post waking

v) 8 hours post waking

vi) 12 hours post waking

They were also asked to complete self-assessments of their mood and sleep behavior. The study lasted for 5 days. Day 1 of the study served as the control day in which saliva samples were collected and questionnaires completed but no treatment regimen had been prescribed. On days 2-5 of the study, the panelists were asked to smell a pleasant relaxing fragrance (PD1861 supplied by Quest International) and while sitting comfortably in room with low ambient lighting and listening to relaxing music (music CD entitled “Relax with Ocean Relaxing Surf” by Eclipse Music Group) for a period of 5 minutes approximately 30 minutes after morning waking, 4 hours after waking and 8 hours after waking. Prior to bedtime on days 2-5 panelists were also asked to take a 15 minute fragranced bath (fragrance PD1861 supplied by Quest International) at approximately 35 C. while listening to relaxing music (music CD entitled “Relax with Ocean Relaxing Surf” by Eclipse Music Group) in a room with low ambient lighting.

The salivary cortisol data for Example 3 Group C is presented in Tables 18 26 below.

TABLE 18
Group C Salivary Cortisol in Sample Collected 30 Minutes Post
Waking

| | Day 1 | Day 2 | Day 3 | Day 4 | Day 5 |
|---|---|---|---|---|---|
| Panelist | (ug/dl) | (ug/dl) | (ug/dl) | (ug/dl) | (ug/dl) |
| C-1 | 0.496 | 0.548 | 0.565 | 0.195 | 0.260 |
| C-2 | 0.178 | 0.092 | 0.104 | 0.177 | 0.238 |
| C-3 | 0.283 | 0.291 | 0.159 | 0.416 | 0.749 |
| C-4 | 0.815 | 0.353 | 0.365 | 0.536 | 0.500 |
| C-5 | 0.658 | 0.981 | 0.724 | 0.861 | 0.728 |
| C-6 | 0.441 | 0.107 | 0.033 | 0.160 | 0.153 |
| C-7 | 0.754 | 0.442 | 0.368 | 0.141 | 0.080 |

TABLE 19
Group C Salivary Cortisol in Sample Collected 4 hours Post Waking

| | Day 1 | Day 2 | Day 3 | Day 4 | Day 5 |
|---|---|---|---|---|---|
| Panelist | (ug/dl) | (ug/dl) | (ug/dl) | (ug/dl) | (ug/dl) |
| C-1 | 0.282 | 0.175 | 0.316 | 0.197 | 0.319 |
| C-2 | 0.116 | 0.088 | 0.096 | 0.040 | 0.050 |
| C-3 | 0.143 | 0.153 | 0.141 | 0.168 | 0.128 |
| C-4 | 0.285 | 0.200 | 0.200 | 0.140 | 0.169 |
| C-5 | 0.561 | 0.319 | 0.275 | 0.237 | 0.506 |
| C-6 | 0.088 | 0.054 | 0.050 | 0.067 | 0.046 |
| C-7 | 0.154 | 0.546 | 0.092 | 0.125 | 0.084 |

TABLE 20
Group C Salivary Cortisol in Sample Collected 8 Hours Post Waking

| | Day 1 | Day 2 | Day 3 | Day 4 | Day 5 |
|---|---|---|---|---|---|
| Panelist | (ug/dl) | (ug/dl) | (ug/dl) | (ug/dl) | (ug/dl) |
| C-1 | 0.137 | 0.105 | 0.079 | 0.145 | 0.242 |
| C-2 | 0.072 | 0.023 | 0.119 | 0.043 | 0.032 |
| C-3 | 0.086 | 0.094 | 0.161 | 0.102 | 0.096 |
| C-4 | 0.326 | 0.189 | 0.176 | 0.157 | 0.108 |
| C-5 | 0.209 | 0.404 | 0.196 | 0.233 | 0.179 |
| C-6 | 0.046 | 0.048 | 0.105 | 0.052 | 0.072 |
| C-7 | 0.087 | NA | 0.037 | 0.038 | 0.084 |

TABLE 21
Group C Salivary Cortisol in Sample Collected 12 Hours Post
Waking

| | Day 1 | Day 2 | Day 3 | Day 4 | Day 5 |
|---|---|---|---|---|---|
| Panelist | (ug/dl) | (ug/dl) | (ug/dl) | (ug/dl) | (ug/dl) |
| C-1 | 0.038 | 0.055 | 0.070 | 0.072 | 0.069 |
| C-2 | 0.032 | 0.022 | 0.029 | 0.020 | 0.060 |
| C-3 | 0.070 | 0.121 | 0.094 | 0.093 | 0.052 |
| C-4 | 0.069 | 0.059 | 0.050 | 0.083 | 0.021 |
| C-5 | 0.094 | 0.092 | 0.109 | 0.134 | 0.088 |
| C-6 | 0.035 | 0.040 | 0.062 | 0.041 | 0.041 |
| C-7 | 0.054 | 0.244 | 0.591 | 0.010 | 0.084 |

TABLE 22
Group C Salivary Cortisol in Sample Collected 30 Minutes Post
Bathing

| | Day 1 | Day 2 | Day 3 | Day 4 | Day 5 |
|---|---|---|---|---|---|
| Panelist | (ug/dl) | (ug/dl) | (ug/dl) | (ug/dl) | (ug/dl) |
| C-1 | 0.045 | 0.840 | 0.037 | 0.046 | 0.061 |
| C-2 | 0.031 | 0.018 | 0.031 | 0.120 | 0.036 |
| C-3 | 0.021 | 0.033 | 0.047 | 0.046 | 0.042 |
| C-4 | 0.227 | 0.026 | 0.040 | 0.037 | 0.048 |
| C-5 | 0.121 | 0.039 | 0.253 | 0.131 | 0.140 |
| C-6 | 0.021 | 0.095 | 0.022 | 0.018 | 0.037 |
| C-7 | NA | 0.209 | 0.207 | 0.039 | 0.085 |

TABLE 23
Group C Mean Cortisol Values

| Minutes Since | | | | | |
|---|---|---|---|---|---|
| Morning | Day 1 | Day 2 | Day 3 | Day 4 | Day 5 |
| Waking | (ug/dl) | (ug/dl) | (ug/dl) | (ug/dl) | (ug/dl) |
| 30 | 0.518 | 0.402 | 0.331 | 0.355 | 0.389 |
| 240 | 0.233 | 0.219 | 0.167 | 0.139 | 0.186 |
| 480 | 0.138 | 0.144 | 0.125 | 0.110 | 0.116 |
| 720 | 0.056 | 0.094 | 0.140 | 0.065 | 0.060 |

Day 1 is the control, while on day 2 the panelist would experience fragrance, relaxing music and low ambient lighting at 3 time points throughout the day, and a bath with a relaxing fragrance (PD1861 from Quest International) coupled with relaxing music “Relax With Ocean Relaxing Surf” by Eclipse Music Group, under low ambient lighting prior to bedtime, which would be repeated through and including Day 5. An integrative measure of cortisol calculated from the area under the curve for each day may be made. The values of the area under the curve (AUC) for Group C for each of the 5 days of the study are presented in Table 24 below.

TABLE 24
Total Area Under Curve of Salivary Cortisol for Group C

| Day | Total AUC (arbitrary units) |
|---|---|
| 1 | 146.7 |
| 2 | 137.3 |
| 3 | 119.1 |
| 4 | 102.8 |
| 5 | 117.7 |

The values of the AUC minus the morning peak area for Group C for each of the 5days of the study are present in Table 25 below.

TABLE 25
AUC Minus the Morning Peak Area of Salivary Cortisol for Group C

| Day | AUC Minus Peak Area (arbitrary units) |
|---|---|
| 1 | 116.7 |
| 2 | 118.1 |
| 3 | 101.9 |
| 4 | 80.1 |
| 5 | 96.4 |

TABLE 26
Group C Mean Cortisol 4 Hours Post Waking

| | Mean Cortisol 4 Hours Post Waking |
|---|---|
| Day | (ug/dl) |
| 1 | 0.233 |
| 2 | 0.219 |
| 3 | 0.167 |
| 4 | 0.139 |
| 5 | 0.186 |

The cortisol data for Group C surprisingly indicates a reduction in cortisol for days 2-5 in comparison to control day 1. Importantly, a reduction in cortisol was found in all of the indices used in this study for investigating HPA activity. This clearly demonstrates that a combination or regimen of sensory stimuli can provide long term and lasting effects on the stress level of the individual, by modifying HPA activity.

It is noted that while the same relaxing fragrance was used throughout the three different cells, and provided a relaxing and pleasing sensation to Groups A and B, no long lasting effect of stress reduction as measured by any of the indices useful in studying HPA activity: total daily cortisol, cortisol minus the morning peak, and the cortisol value approximately 4 hours post waking was observed. These examples clearly demonstrate that there is a difference between a momentary, pleasing effect, and a long lasting effect that can reduce one's stress level.

### Example 5

### Downregulation OF HPA Activity Reduces Stress in Individuals

The effects of stress are diverse and can manifest itself differently among a group of individuals. Questionnaires that aim to subjectively evaluate stress levels in individuals usually look at a range of parameters including mood, behavior and somatic symptoms. These parameters are looked at globally to assess the stress level of the individual. Individual panelists were asked to rate their physical, energy, emotional and stress levels, before and after the 5-day study. The results of the analysis of the questionnaires are presented in Tables 27 and 28 below.

TABLES 27 and 28
Questionnaire data for groups A, B, C (Examples 1, 2, 3)

| | % PANELISTS WHO REPORTED AN IMPROVEMENT AT | | | | |
|---|---|---|---|---|---|
| | END OF STUDY | | | | |
| | | | | | GROUP |
| GROUP | PHYSICAL | ENERGY | EMOTIONAL | STRESS | MEAN |
| A | 37.5 | 25 | 50 | 75 | 46.9 |
| B | 37.5 | 37.5 | 25 | 25 | 31.3 |
| C | 37.5 | 62.5 | 62.5 | 75 | 59.4 |

| | SIGNIFICANT IMPROVEMENT BY THE END OF | | | |
|---|---|---|---|---|
| | STUDY Y/N (p < 0.1) | | | |
| GROUP | PHYSICAL | ENERGY | EMOTIONAL | STRESS |
| A | N | N | N | Y |
| B | Y | N | N | N |
| C | N | N | Y | Y |

Use of a self-assessment questionnaire which graded mood and somatic symptoms at the beginning and at the end of the five day study period for Groups A, B and C in Examples 2, 3 and 4 respectively, showed that while all groups reported some benefit in mood and somatic parameters, the greatest global improvements were seen for Group C, Example 4.

The results indicate that a pleasant experience, which had a relatively short-term effect on HPA activity, does result in an improvement in mood and somatic symptoms associated with stress. However, the greatest improvements in mood and somatic symptoms associated with stress were found for Group C, Example 4, which had significant downregulation in HPA activity.

The results of the subjective self-evaluation are consistent with the results in the objective physiological assessments of HPA activity, in that group C experienced the greatest down regulation of HPA activity. These results indicate that downregulation of HPA activity leads to a user perceivable reduction in stress.

### Example 6

### Topical Application of an Unfragranced Lotion in 2-Week Dermatologist Controlled Skin-Care Study

A group of 12 panelists of either sex in the age range of 13 to 40 years were asked to participate in a two week long skin care study in which they would be required to consult with a dermatologist who would prescribe a topical skin care product for daily application. The skin care product hat was applied to the panelists was Clean & Clear Persa-Gel 5%, without the presence of the benzoyl peroxide. The panelists were required to collect saliva samples for the purposes of measuring cortisol. Saliva samples were collected on the first day of the study, prior to any treatment in order to assess baseline cortisol values and subsequent samples were collected one and two weeks later. Panelists collected 1 ml of saliva by drooling or spitting into independent vials on each of the three days that samples were required at the following time points:

i) upon waking

ii) 30 minutes post waking

iii) 65 minutes post waking

iv) 4 hours post waking

v) 8 hours post waking

vi) 12 hours post waking

The salivary cortisol data collected for the study group (group D) is presented in Tables 29-38 below. The cortisol concentration data reported in examples 1 to 5 were in units of ug/dl, whereas the units of concentration of cortisol reported in examples 6 and 7 are in nmol/l. For comparison purposes, 1 ug/dl is equivalent to 27.6 nmol/l.

TABLE 29
Group D Saliva Sample Collected Upon Waking

| | Baseline | Week One | Week Two |
|---|---|---|---|
| | Cortisol | Cortisol | Cortisol |
| Panelist | (nmol/l) | (nmol/l) | (nmol/l) |
| D1 | 21.2 | 11.7 | 28.3 |
| D2 | 13.1 | 10.1 | 7.2 |
| D3 | 17.7 | 9.4 | 24.5 |
| D4 | 19.6 | 11.5 | 21.4 |
| D5 | 2.3 | 15.3 | 3.00 |
| D6 | 12.7 | 35.7 | 13.4 |
| D7 | 17.8 | 8.8 | 7.7 |
| D8 | 10.8 | 25.1 | 6.8 |
| D9 | 7.9 | 1.6 | 45.5 |
| D10 | 8.7 | 1.7 | NA |
| D11 | 17.5 | 45.8 | NA |
| D12 | 1.9 | 7.8 | NA |

TABLE 30
Group D Saliva Sample Collected 30 Minutes Post Waking

| | Baseline | Week One | Week Two |
|---|---|---|---|
| | Cortisol | Cortisol | Cortisol |
| Panelist | (nmol/l) | (nmol/l) | (nmol/l) |
| D1 | 43.2 | 10.7 | 45.9 |
| D2 | 9.8 | 19.7 | 0.7 |
| D3 | 85.6 | 10.2 | 20.7 |
| D4 | 23.4 | 7.3 | 15.8 |
| D5 | 8.7 | 20.2 | 2. |
| D6 | 5.1 | 14.7 | 10.3 |
| D7 | 9.2 | 4.6 | 8.4 |
| D8 | 17.6 | 9.4 | 7.4 |
| D9 | 79.2 | 1.1 | 39.0 |
| D10 | 6.2 | 0.8 | NA |
| D11 | 12.0 | 36.2 | NA |
| D12 | 15.1 | 23.7 | NA |

TABLE 31
Group D Saliva Sample Collected 65 Minutes Post Waking

| | Baseline | Week One | Week Two |
|---|---|---|---|
| | Cortisol | Cortisol | Cortisol |
| Panelist | (nmol/l) | (nmol/l) | (nmol/l) |
| D1 | 13.6 | 5.9 | 36.2 |
| D2 | 3.4 | 10.8 | 1.6 |
| D3 | 33.4 | 10.1 | 18.2 |
| D4 | 17.4 | 6.7 | 12.8 |
| D5 | 5.1 | 26.8 | 2.4 |
| D6 | 10.0 | 16.6 | 17.0 |
| D7 | 16.3 | 10.2 | 7.8 |
| D8 | 14.1 | 23.2 | 4.9 |
| D9 | 8.1 | 21.8 | 85 |
| D10 | 6.7 | 1.5 | NA |
| D11 | 6.7 | 6.5 | NA |
| D12 | 12.0 | NA | NA |

TABLE 32
Group D Saliva Sample Collected 4 Hours Post Waking

| | Baseline | Week One | Week Two |
|---|---|---|---|
| | Cortisol | Cortisol | Cortisol |
| Panelist | (nmol/l) | (nmol/l) | (nmol/l) |
| D1 | 12.1 | 8.2 | 16.7 |
| D2 | 2.6 | 5.1 | 1.0 |
| D3 | 8.3 | 9.4 | 5.7 |
| D4 | 10.6 | 7.3 | 21.8 |
| D5 | 12.1 | 10.6 | 1.6 |
| D6 | 10.5 | 36.1 | 10.6 |
| D7 | 15.9 | 4.9 | 10.4 |
| D8 | 11.5 | 6.4 | 8.5 |
| D9 | 11.9 | 1.2 | 3.1 |
| D10 | 4.0 | 1.1 | NA |
| D11 | 5.4 | 6.0 | NA |
| D12 | 21.9 | NA | NA |

TABLE 33
Group D Saliva Sample 8 Hours Post Waking

| | Baseline | Week One | Week Two |
|---|---|---|---|
| | Cortisol | Cortisol | Cortisol |
| Panelist | (nmol/l) | (nmol/l) | (nmol/l) |
| D1 | 4.9 | 1.7 | 28.0 |
| D2 | 12.8 | 3.5 | 4.2 |
| D3 | 17.5 | 8.7 | 6.5 |
| D4 | 11.3 | 5.5 | 10.8 |
| D5 | 7.7 | 14.8 | 0.7 |
| D6 | 16.7 | 16.4 | 6.4 |
| D7 | 7.3 | 9.9 | 8.0 |
| D8 | 15.7 | 16.3 | 9.7 |
| D9 | 4.7 | 1.6 | 5.5 |
| D10 | 1.0 | 1.1 | NA |
| D11 | 4.9 | 19.8 | NA |
| D12 | 3.6 | NA | NA |

TABLE 34
Group D Saliva Sample 12 Hours Post Waking

| | Baseline | Week One | Week Two |
|---|---|---|---|
| | Cortisol | Cortisol | Cortisol |
| Panelist | (nmol/l) | (nmol/l) | (nmol/l) |
| D1 | 1.3 | 3.3 | 46.0 |
| D2 | 11.1 | 0.6 | 1.6 |
| D3 | 1.0 | 9.1 | 2.8 |
| D4 | 13.7 | 1.6 | 11.8 |
| D5 | 7.5 | 13.0 | 1.1 |
| D6 | 4.8 | 15.7 | 32.3 |
| D7 | 0.1 | 5.9 | 8.8 |
| D8 | 4.8 | 2.0 | 8.3 |
| D9 | 11.7 | 18.1 | 1.4 |
| D10 | 3.5 | 0.7 | NA |
| D11 | 2.3 | 3.2 | NA |
| D12 | 7.1 | NA | NA |

TABLE 35
Group D Mean Cortisol Values

| | Baseline | | |
|---|---|---|---|
| | Mean | Week One | Week Two |
| Minutes since | Cortisol | Mean Cortisol | Mean Cortisol |
| waking | (nmol/l) | (nmol/l) | (nmol/l) |
| 30 | 25.5 | 13.2 | 16.7 |
| 240 | 9.9 | 8.8 | 8.8 |
| 480 | 9.0 | 9.0 | 8.9 |
| 720 | 5.8 | 6.6 | 12.6 |

The values of the total AUC, mean AUC minus morning peak and Mean Cortisol 4 Hours Post Waking for Group D at baseline and after one and two weeks of treatment are presented in Tables 36, 37 and 38 respectively below.

TABLE 36
Group D Mean AUC of Salivary Cortisol

| | Mean AUC (arbitrary unit) |
|---|---|
| Baseline | 7770 |
| Week 1 | 6320 |
| Week 2 | 7390 |

TABLE 37
Mean AUC minus morning peak for Salivary Cortisol Group D

| | Mean AUC minus morning peak (arbitrary units) |
|---|---|
| Baseline | 6130 |
| Week 1 | 5850 |
| Week 2 | 6560 |

TABLE 38
Group D Mean Cortisol 4 Hours Post Waking

| | Mean Cortisol 4 Hours Post Waking (nmol/l) |
|---|---|
| Baseline | 9.9 |
| Week 1 | 8.8 |
| Week 2 | 8.8 |

The cortisol data for Example 5, Group D, indicates that the total amount of daily cortisol is lower than baseline at weeks 1 and 2, and a small reduction from baseline in 4 hour post waking cortisol was observed at weeks 1 and 2. Further, a small reduction from baseline in the value of the AUC minus the morning peak was observed at week one, but by week 2 this value had increased above baseline.

### Example 7

### Two Week Long Dermatologist Controlled Skin Care Study With Daily Sensory Regimen

A group of 12 panelists of either sex in the age range of 13 to 40 years were asked to participate in a two week long skin care study in which they were required to consult with a dermatologist who prescribed a daily sensory regimen consisting of smelling a pleasant, relaxing fragrance (PD1861 supplied by Quest International), listening to relaxing music (music CD entitled “Relax with Ocean Relaxing Surf” by Eclipse Music Group), low ambient lighting and bathing regimen before bedtime in which panelists were asked to take a 15 minute fragranced oath (fragrance PD1861 supplied by Quest International) at approximately 35 C. while listening to relaxing music (music CD entitled “Relax with Ocean Relaxing Surf” by Eclipse Music Group) in a room with low ambient lighting.

The panelists were required to collect saliva samples for the purposes of measuring cortisol. Saliva samples were collected on the first day of the study, prior to any treatment in order to assess baseline cortisol values and subsequent samples were collected one and two weeks later. Panelists collected 1 ml of saliva by drooling or spitting into independent vials on each of the three days that samples were required at the following timepoints:

Saliva samples were collected at the following timepoints on those days:

i) upon waking

ii) 30 minutes post waking

iii) 65 minutes post waking

iv) 4 hours post waking

v) 8 hours post waking

vi) 12 hours post waking

They were also asked to complete self-assessments of their mood and other symptoms related to their skin condition. The study lasted for 2 weeks. Day 1 of the study served as the baseline in which saliva samples were collected and questionnaires completed but no treatment regimen had been prescribed. On the remaining days of the study, the panelists were asked to smell a pleasant relaxing fragrance (PD1861 supplied by Quest International) while seated comfortably in room with low ambient lighting and listening to relaxing music music (music CD entitled “Relax with Ocean Relaxing Surf” by Eclipse Music Group) for a period of 5 minutes approximately 30 minutes after morning waking, 4 hours after waking and 8 hours after waking. Prior to bedtime on days 2-5 panelists were also asked to take a 15 minute fragranced bath (fragrance PD 1861 supplied by Quest International) at approximately 35 C. while listening to relaxing music (music CD entitled “Relax with Ocean Relaxing Surf” by Eclipse Music Group) in a room with low ambient lighting.

The salivary cortisol data for Example 7 Group E is presented in Tables 39-48 below:

TABLE 39
Group E Salivary Cortisol in Sample Collected Upon Waking

| | Baseline | Week One | Week Two |
|---|---|---|---|
| | Cortisol | Cortisol | Cortisol |
| | (nmol/l) | (nmol/l) | (nmol/l) |
| E1 | 28.9 | 18.9 | 28.1 |
| E2 | 36.8 | 23.2 | 6.6 |
| E3 | 33.9 | 20.8 | 13.9 |
| E4 | 19.2 | 23.8 | 21.5 |
| E5 | 9.0 | 5.0 | 14.6 |
| E6 | 7.8 | 39.9 | 15.0 |
| E7 | 29.2 | 19.6 | 17.2 |
| E8 | 18.4 | 13.2 | 9.1 |
| E9 | 5.2 | 29.2 | NA |
| E10 | 6.6 | 29.7 | NA |
| E11 | 23.7 | 10.1 | NA |
| E12 | 14.7 | 4.3 | NA |

TABLE 40
Group E Salivary Cortisol in Sample Collected 30 Minutes Post
Waking

| | Baseline | Week One | Week Two |
|---|---|---|---|
| | Cortisol | Cortisol | Cortisol |
| | (nmol/l) | (nmol/l) | (nmol/l) |
| E1 | 11.2 | 22.5 | 52.0 |
| E2 | 33.0 | 21.7 | 5.9 |
| E3 | 46.2 | 63.2 | 56.9 |
| E4 | 23.3 | 12.8 | 21.1 |
| E5 | 24.0 | 5.8 | 10.4 |
| E6 | 19.2 | 8.6 | 11.1 |
| E7 | 37.3 | 6.1 | 26.8 |
| E8 | 26.6 | 22.6 | 10.7 |
| E9 | 4.7 | 24.2 | NA |
| E10 | 14.9 | 48.4 | NA |
| E11 | 20.5 | 14.6 | NA |
| E12 | 12.4 | 6.1 | NA |

TABLE 41
Group E Salivary Cortisol in Sample Collected 65 Minutes Post
Waking

| | Baseline | Week One | Week Two |
|---|---|---|---|
| | Cortisol | Cortisol | Cortisol |
| | (nmol/l) | (nmol/l) | (nmol/l) |
| E1 | 34.4 | 12.6 | 11.4 |
| E2 | 21.8 | 31.0 | 5.6 |
| E3 | 49.5 | 39.6 | 13.0 |
| E4 | 18.9 | 7.0 | 9.4 |
| E5 | 55.4 | 13.0 | 19.2 |
| E6 | 10.5 | 6.9 | 17.3 |
| E7 | 25.9 | 3.5 | 32.8 |
| E8 | 29.0 | 18.5 | 8.1 |
| E9 | 8.4 | 12.5 | NA |
| E10 | 5.0 | 2.1 | NA |
| E11 | 16.7 | 8.9 | NA |
| E12 | 6.7 | 4.5 | NA |

TABLE 42
Group E Salivary Cortisol in Sample Collected 4 Hours Post Waking

| | Baseline | Week One | Week Two |
|---|---|---|---|
| | Cortisol | Cortisol | Cortisol |
| | (nmol/l) | (nmol/l) | (nmol/l) |
| E1 | 26.0 | 6.0 | 6.9 |
| E2 | 6.7 | 1.8 | 7.1 |
| E3 | 11.8 | 3.4 | 9.1 |
| E4 | 7.4 | 5.0 | 12.2 |
| E5 | 35.9 | 17.9 | 29.1 |
| E6 | 6.9 | 12.5 | 7.2 |
| E7 | 3.8 | 5.3 | 8.1 |
| E8 | 9.2 | 6.3 | 4.7 |
| E9 | 5.0 | 4.1 | NA |
| E10 | 21.5 | 13.5 | NA |
| E11 | 11.5 | 6.8 | NA |
| E12 | 6.9 | 21.8 | NA |

TABLE 43
Group E Salivary Cortisol in Sample Collected 8 Hours Post Waking

| | Baseline | Week One | Week Two |
|---|---|---|---|
| | Cortisol | Cortisol | Cortisol |
| | (nmol/l) | (nmol/l) | (nmol/l) |
| E1 | 17.0 | 5.3 | 5.1 |
| E2 | 3.4 | 5.1 | 5.3 |
| E3 | 8.6 | 5.1 | 10.4 |
| E4 | 4.0 | 8.1 | 7.0 |
| E5 | 11.3 | 8.3 | 4.5 |
| E6 | 15.9 | 1.0 | 19.9 |
| E7 | 7.5 | 11.5 | 24.9 |
| E8 | 7.8 | 8.8 | 30.3 |
| E9 | 6.7 | 2.5 | NA |
| E10 | 45.7 | 11.6 | NA |
| E11 | 12.5 | 14.9 | NA |
| E12 | 8.4 | 2.9 | NA |

TABLE 44
Group E Salivary Cortisol in Sample Collected 12 Hours Post
Waking

| | Baseline | Week One | Week Two |
|---|---|---|---|
| | Cortisol | Cortisol | Cortisol |
| | (nmol/l) | (nmol/l) | (nmol/l) |
| E1 | 8.3 | 11.6 | 17.2 |
| E2 | 5.0 | 2.7 | 2.8 |
| E3 | 7.1 | 2.1 | 1.4 |
| E4 | 9.5 | 3.11 | 1.8 |
| ES | 9.5 | 7.6 | 4.2 |
| E6 | 10.3 | 0.9 | 4.5 |
| E7 | 13.8 | 1.7 | 12.9 |
| E8 | 5.3 | 3.9 | 2.8 |
| E9 | 15.0 | 7.1 | NA |
| E10 | 3.0 | 4.8 | NA |
| E11 | 14.4 | 3.6 | NA |
| E12 | 6.1 | 8.6 | NA |

TABLE 45
Group E Salivary Cortisol in Sample Collected 30 Minutes Post
Bathing

| | Baseline | Week One | Week Two |
|---|---|---|---|
| | Cortisol | Cortisol | Cortisol |
| | (nmol/l | (nmol/l) | (nmol/l) |
| E1 | No sample | 2.7 | 16.1 |
| | collected | | |
| E2 | No sample | 6.4 | 11.8 |
| | collected | | |
| E3 | No sample | 2.6 | 1.3 |
| | collected | | |
| E4 | No sample | 1.6 | 3.9 |
| | collected | | |
| E5 | No sample | 3.8 | 3.4 |
| | collected | | |
| E6 | No sample | 3.0 | 10.9 |
| | collected | | |
| E7 | No sample | 1.1 | 1.4 |
| | collected | | |
| E8 | No sample | 5.1 | 8.2 |
| | collected | | |
| E9 | No sample | 7.4 | NA |
| | collected | | |
| E10 | No sample | 1.4 | NA |
| | collected | | |
| E11 | No sample | 4.0 | NA |
| | collected | | |
| E12 | No sample | 3.6 | NA |
| | collected | | |

TABLE 46
Group E Mean Cortisol Values

| | | Week one | week two |
|---|---|---|---|
| | baseline mean | mean | mean |
| Minutes since | Cortisol | Cortisol | Cortisol |
| waking | (nmol/l) | (nmol/l) | (nmol/l) |
| 30 | 22.8 | 21.4 | 24.4 |
| 240 | 12.7 | 8.7 | 10.6 |
| 480 | 12.4 | 7.1 | 13.4 |
| 720 | 8.9 | 4.8 | 6.0 |

Day 1 is the control; day 2 is fragrance in the morning. Days 2-5 no treatment. An integrative measure of cortisol calculated from the area under the curve for each day may be made. The values of the area under the curve (AUC) for Group E for each of the 5 days of the study are presented in Table 45 below.

TABLE 47
Total Area Under Curve of Salivary Cortisol for Group E

| | Total AUC |
|---|---|
| Baseline | 9300 |
| Week 1 | 5780 |
| Week 2 | 7210 |

The values of the AUC minus the peak are for Group E for baseline and weeks 1 and 2 of the study are presented in Table 46 below.

TABLE 48
AUC Minus the Morning Peak Area for Salivary Cortisol for Group
E

| | Total AUC Minus the Peak Area |
|---|---|
| Baseline | 8250 |
| Week 1 | 4450 |
| Week 2 | 5760 |

The mean values of cortisol 4 hours post waking for Group E for baseline and weeks 1 and 2 of the study are presented in Table 49 below.

TABLE 49
Group Mean Cortisol 4 Hours Post Waking

| | Mean Cortisol 4 Hours Post Waking |
|---|---|
| | Cortisol |
| | (nmol/l) |
| Baseline | 12.7 |
| Week 1 | 8.7 |
| Week 2 | 10.6 |

### Example 8

### Downregulation of HPA Activity Improves the Quality of Life of Individuals

The Quality of Life of an individual may be studied by use of validated questionnaires which allow study investigators to quantify how a treatment effects the quality of life of an individual coping with a condition or situation in their life. Skindex is a quality of life questionnaire used in the field of dermatology to quantify the effect of a skin condition on the Quality of Life of the individual suffering from the condition and enables study investigators to quantify how a treatment or intervention used by the individual suffering from the skin condition, effects the Quality of Life of the individual and is described in Chren, Mary-Margarel, Lasek, Rebecca J., Flocke, Susan A., Zyzanski, Stephen J. “Improved Discriminative and Evaluative Capability of a Refined Version of Skindex, a Quality-of-Life instrument for Patients with Skin Diseases” 1997, Arch Dermatol, 133, 1433-1440, the disclosure of which is hereby incorporated by reference.

Groups D and E from Examples 6 and 7 respectively, completed the Skindex questionnaire at baseline and weeks 1 and 2 of the study. The aim of this use of this questionnaire was to determine how the downregulation of HPA activity induced by the treatment regimes effected the Quality of Life of the individuals participating in the study. Change in each parameter of the questionnaire was considered to be significant with a p value less than 0.05.

The results are presented in table 50 below.

TABLE 50
Improvement in Quality of Life: Points Improvement in Skindex
Questionnaire Rating

| | Significant Improvement | Significant Improvement |
|---|---|---|
| Skindex Category | Group D Example 6 | Group E Example 7 |
| Symptomatic | No | No |
| Functional | No | Yes |
| Emotional | Yes | Yes |
| Overall | No | Yes |

The results indicate that the Quality of Life of an individual may be improved by downregulation of the HPA axis.

While the example here relates to the Quality of Life of an individual suffering from a skin condition, the downregulation of the HPA axis leading to an improvement on the Quality of Life on an individual is not limited to this example. it is obvious to one of normal skill in the art that downregulation of the HPA axis as a means to improving the Quality of Life of an individual applies to individuals coping with any problem, condition or stressful situation which has a detrimental effect on the individuals Quality of Life.

### Summary Of Effects of Treatment Regimens for Examples 2, 3, 4, 6 and 7 on HPA Activity

A summary of the effects of each of the treatments in Examples 2, 3, 4, 6, and 7 on HPA activity are presented in Tables 51 and 52 below.

TABLE 51
Summary of Indices of HPA activity: Effect of treatments on HPA
activity for groups A, B and C

| | | Percentage change | |
|---|---|---|---|
| | Percentage change | from | percentage change |
| | from | control/baseline | cortisol value |
| | control/baseline | AUC | 4 hours |
| Group | AUC | minus am peak | post waking |
| A | 26.3 | 20.4 | 66.7 |
| B | 141.9 | 212.6 | 99.3 |
| C | −19.7 | −17.4 | −20.1 |

The results of the HPA activity analyses for groups A, B and C, Examples 2, 3, and 4 respectively summarized in Table 51 clearly demonstrates the effectiveness of the regimen practiced by group C in downregulating the activity of the HPA axis. Further Example 5 demonstrated that this downregulation in HPA activity correlated with a reduction in self assessed global parameters of stress.

TABLE 52
Summary of Indices of HPA activity: Effect of treatments on HPA
activity for groups D an E

| | | | Percentage change | | percentage change | |
|---|---|---|---|---|---|---|
| | Percentage change | | from control/ | | cortisol value | |
| | from control/ | | baseline AUC | | 4 hours post | |
| | baseline AUC | | minus am peak | | waking | |
| Group | Week 1 | week 2 | Week 1 | Week 2 | week 1 | week 2 |
| D | −18.6 | −4.8 | −4.6 | −7 | −11.6 | −11 |
| E | −37.8 | −22.5 | −46 | −30.1 | −31.6 | −17 |

The results of the HPA activity analyses for groups D and E summarized in Table 52, Examples 6 and 7 respectively, is summarized in table 52 and indicates that both groups experienced a downregulation in HPA activity, and that the greatest downregulation was observed for the group practicing the sensory regimen, group E. Further, the downregulation of HPA activity observed for both groups was sufficient to lead to an improvement in the Quality of Life of the individuals participating in the study, as was demonstrated in Example 8.

What is claimed is:

1. A method for reducing chronic stress in a mammal by downregulating the activity of the HPA axis by administering to said mammal an effective amount of a sensory regimen.

2. A method according to claim 1, wherein the sensory regimen includes the administration of a CRH antagonist or an antidepressant.

3. A method according to claim 1, wherein the activity of the HPA axis is downregulated by reducing the amount of total daily adrenocortical hormone in said mammal.

4. The method of claim 3, wherein the total daily adrenocortical hormone is cortisol.

5. The method of claim 4, wherein cortisol is reduced by at least about 5 to about 50%, based on the total daily cortisol present, in said mammal at the start of said regimen.

6. The method of claim 4, wherein cortisol is reduced by at least about 10 to about 40%, based on the total daily cortisol present in said mammal at the start of said regimen.

7. The method of claim 4, wherein cortisol is reduced by at least about 15 to about 30%, based on the total daily cortisol present in said mammal at the start of said regimen.

8. The method of claim 3, wherein the reduction occurs within a period of about 1 to about 14 days from the start of the regimen.

9. The method of claim 8, wherein the reduction is maintained for a period of 1 day to 2 years.

10. A method of improving the quality of life of an individual comprising reducing chronic stress according to the method of claim 1.

11. A method according to claim 1, wherein the activity of the HPA axis is downregulated by reducing adrenocortical hormone in said mammal at any time point in the period from about 4 to about 8 hours following morning waking.

12. A method according to claim 11, wherein the adrenocortical hormone is cortisol.

13. The method of claim 12, wherein cortisol is reduced by about, 5 to about 60%, based on the amount of cortisol present in said mammal at the start of said regimen.

14. The method of claim 12. wherein cortisol is reduced by about 10 to about 60%, based on the amount of cortisol present in said mammal at the start of said regimen.

15. The method of claim 12, wherein cortisol is reduced by about 20 to about 50%, based on the amount of cortisol present in said mammal at the start of said regimen.

16. The method of claim 11, wherein the reduction occurs within a period of 1 to 14 days from the start of the regimen

17. The method of claim 16, wherein the reduction is maintained for a period of 1 day to 2 years.

18. A method according to claim 1, wherein the activity of the HPA axis is downregulated by reducing the total daily adrenocortical hormone minus the integrative measure of morning peak adrenocortical hormone in said mammal.

19. The method of claim 18, wherein the adrenocortical hormone is cortisol.

20. The method of claim 19, wherein cortisol is reduced by about 5 to about 70%, based on the amount of cortisol present in said mammal at the start of said regimen.

21. The method of claim 19, wherein cortisol is reduced by about 10 to about 60%, eased on the amount of cortisol present in said mammal at the start of said regimen.

22. She method of claim 19, wherein cortisol is reduced by about 20 to about 50%, based on the amount of cortisol present in said mammal at the start of said regimen.

23. The method of claim 18, wherein the reduction occurs within a period of 1 to 14days from the start of the regimen.

24. The method of claim 23, wherein the reduction is maintained for a period of f day to 2 years.
